(19) United States
(12) Patent Application Publication
(10) Pub. No.: US 2017/0354422 A1
(43) Pub. Date: Dec. 14, 2017

(54) EMERGENCY ARTERIAL TOURNIQUET DEVICE AND ASSOCIATED METHODS OF USE AND MANUFACTURE

(71) Applicant: Hawaii Product Development, Inc., Honolulu, HI (US)

(72) Inventor: Darrin Brub, Honolulu, HI (US)

(73) Assignee: Hawaii Product Development, Inc., Honolulu, HI (US)

(21) Appl. No.: 15/669,597

(22) Filed: Aug. 4, 2017

Related U.S. Application Data
(63) Continuation-in-part of application No. 13/397,166, filed on Feb. 15, 2012, now Pat. No. 9,750,507.
(60) Provisional application No. 61/444,487, filed on Feb. 18, 2011.

(51) Int. Cl. A61B 17/132

(52) U.S. Cl. CPC A61B 17/1327

(57) ABSTRACT

Tourniquet devices configured in accordance with several embodiments of the disclosure can include, for example, a strap having a proximal end portion and a distal end portion, a buckle assembly connected to the proximal end portion, and a pulley assembly movably coupled to the strap. Tourniquet devices configured in accordance with the technology can be easily applied because the pulley assemblies can reduce the force a user must exert on the tourniquet devices to constrict or occlude blood flow at a specific artery. Additionally, tourniquet devices disclosed herein can also serve as functional, wearable items (e.g., a belt, integrated into clothing) such that the tourniquet devices are conveniently stored and readily accessible in emergencies.

### CROSS REFERENCES

This application claims priority from U.S. Provisional Patent Application No. 61/444,487 filed Feb. 18, 2011 incorporated herein in its entirety by this reference, via co-pending U.S. Utility Application Ser. No. 13/397,166 filed on Feb. 15, 2012, in the name of the same inventor, the entirety of which is also incorporated herein by this reference.

### TECHNICAL FIELD

The present technology is related to medical devices. In particular, the present disclosure is related to arterial tourniquet devices and associated methods of use and manufacture.

### BACKGROUND

Tourniquets are devices that can control venous and arterial circulation to extremities. In operation, tourniquets apply pressure circumferentially upon skin and underlying tissues of a limb, and in addition apply direct pressure to the target vein or artery. The pressure, circumferential and targeted, transfers to the walls of vessels, causing them to compress, constrict, and become temporarily occluded. To avoid further injury to the limb, the tourniquet pressure should be sufficient to stop blood flow, but should not be strong enough to damage tissue, nerves, and/or blood vessels. The tourniquet should be easily tightenable and loosenable both.

In general, tourniquets are categorized as either surgical tourniquets or emergency tourniquets. Tourniquets used during surgery allow surgeons to work on limbs in a bloodless setting, and accordingly include specialized instruments to gauge the correct pressures for specific applications. Surgical tourniquets, for example, are generally pneumatic devices that include pressure gauges, electrical inflation mechanisms, sensors, and/or outer devices suitable for hospital settings. In contrast, tourniquets used in emergency settings are used to prevent severe blood loss prior to a victim reaching a state of shock and prior to hospital care. Thus, emergency tourniquets are configured to facilitate easy and quick application to stop blood flow from a limb prior to more intensive hospital care.

Emergency tourniquets are especially advantageous in military settings, where pre-hospital care is critical. During combat, for example, explosions (e.g., improvised explosive devices) and gun fire can result in traumatic injuries that require immediate occlusion of blood flow. Additionally, formal medical care may not be readily available in combat situations. Thus, emergency tourniquets are often stored on military personnel and/or in military vehicles to ensure tourniquets are available when traumatic bleeding injuries occur. However, military personnel generally carry heavy gear (e.g., weaponry, backpacks, water containers, etc.) when in combat, making the additional storage of a tourniquet inconvenient. Additionally, tourniquets stored in military vehicles may be difficult to access after an injury causing event because the vehicle may be difficult to reach and/or retrieval of the tourniquet may be impractical due to damage to the vehicle.

In addition, emergency tourniquets are necessary for emergency workers and first responders such as firemen, EMTs, law enforcement personnel, private contractors, and anyone working or playing in a dangerous situation, such as saw-mills, foot patrols, isolated work places and so on.

### BRIEF SUMMARY OF THE INVENTION

The present invention specifically addresses and alleviates the above mentioned deficiencies, more specifically, the present invention is directed to a simple, fast and effective arterial tourniquet device that can be quickly deployed and used in the field, including by the wounded soldier if need be. The arterial tourniquet devices and methods of use according to the present invention are configured in embodiments that include and comprise, for example, a strap having a proximal end portion and a distal end portion, a buckle assembly connected to the proximal end portion, and a pulley assembly movably coupled to the strap. The two pulley assemblies combine to form a single “true pulley” offering mechanical advantage of 2:1 on force applied by a user. In addition, the two pulley assemblies may have a tray/support part which is forced into the limb at a specific point and thus allow a user to rotate the tourniquet device about the limb, line up one pulley assembly over an artery or vein in a calf, thigh, arm, etc, and then tighten it to directly pressure the blood vessel and occlude it. Tourniquet devices configured in accordance with the technology can be easily applied because the pulley assemblies can reduce the force a user must exert on the tourniquet devices to constrict or occlude blood flow, in fact, halving the pressure to be applied. Additionally, tourniquet devices disclosed herein can also serve as functional, wearable items (e.g., a belt, integrated into clothing) such that the tourniquet devices are conveniently stored and readily accessible in emergencies and have multiple other field uses, including, e.g., gun straps, backpack straps, seat belts and as restraint devices, either on personnel or on vehicles, equipment, installations, buildings and so on.

Accordingly, in a first aspect, the present invention provides a tourniquet device comprising a strap having a proximal end portion and a distal end portion, a buckle assembly connected to the proximal end portion of the strap, and a pulley assembly slidably coupled to the strap and movable between the proximal end portion and the distal end portion, the pulley assembly having a support member and a pulley member rotatably coupled to the support member. In a preferred embodiment, the strap has a width sufficient to apply pressure to constrict blood flow, while preventing constricting blood flow to the point of injury or amputation. In another preferred embodiment, one pulley member has a support which is actually forced into the encircled limb when pressure is applied, so as to directly pressure a desired spot, i.e. a blood vessel.

In a further aspect, the buckle assembly a frame for connecting the end portion of the strap, and in a preferred embodiment, can include a second pulley member coupled to the frame to bias the buckle assembly away from the injured limb.

In another aspect, the size of the plurality of openings is proportional to the weight of the flexible material.

In yet a further aspect, the pulley mechanism includes a clamping member to allow free movement of the strap in one direction and restricted movement in the opposite direction.

In a further aspect, the pulley attachment mechanism allows for incremental loosening and tightening of the tourniquet device and selective reattachment to the distal end of the strap as pressure in increased or decreased.

In another aspect of the present invention, a method of applying a tournaquet devices according to the present invention is provided, comprising the steps of encircling a limb above the point of blood loss with a tourniquet device comprising:

a strap having a proximal end portion and a distal end portion;

a buckle assembly connected to the proximal end portion of the strap; and

a pulley assembly slidably coupled to the strap and movable between the proximal end portion and the distal end portion, the pulley assembly having a support member and a pulley member rotatably coupled to the support member; and looping the distal end portion of the strap through the buckle assembly to reverse directions, looping the distal end portion of the strap through the pulley member, applying sufficient pressure to constrict blood flow by pulling the distal end of the strap away from the pulley assembly to tension the strap, transferring circumferential pressure to the limb and direct pressure to any artery below the pulley support, and releasably securing the distal end portion to an attachment mechanism.

It is therefore on embodiment of the invention, in addition to those objectives and aspects mentioned previously, to provide a tourniquet device, for a primary use applying pressure to an artery in a patient's limb and a secondary use worn as a belt looped around a wearer's torso, the tourniquet device comprising:

a strap having a proximal end portion and a distal end portion;

a first buckle assembly connected to the proximal end portion of the strap;

a second pulley assembly slidably coupled to the strap and movable between the proximal end portion and the distal end portion, the second pulley assembly having first and second apertures passing therethrough, the second pulley assembly located at a distance from the first buckle assembly;

the second pulley assembly having a pulley assembly support and a first pulley member rotatably coupled to the pulley assembly support and biased away from the pulley assembly support;

the first buckle assembly having a frame for connection to the proximal end portion of the strap and forming an opening wherein at least one post extends across the opening;

the first buckle assembly further having a second pulley member rotatably coupled to the frame, the second pulley member rotating away from the frame;

the strap passing through the first and second apertures of the second pulley assembly and then about the second pulley of the first buckle assembly;

whereby a pulley having a two-to-one force multiplication is formed by the first and second pulleys and the strap passing between them;

and further whereby an opening is formed through which in a first such usage, such artery in such limb may extend and be encircled and in a second such usage such tourniquet device may be worn as a belt.

It is therefore another embodiment, aspect, objective, and advantage of the present invention to provide a tourniquet device, wherein the first pulley extends away from the pulley assembly support and the opening; whereby when tightened it exerts torque which forces the pulley assembly support into a part of the limb directly under the first pulley, and when the part of the limb directly under the first pulley is such artery, such artery is occluded.

It is therefore another embodiment, aspect, objective, and advantage of the present invention to provide a tourniquet device, wherein the second pulley member includes a pulley reinforcement support extending across the opening and provides support for the second pulley member.

It is therefore another embodiment, aspect, objective, and advantage of the present invention to provide a tourniquet device, wherein the pulley assembly support is configured to include at least one aperture for slidably attaching the pulley assembly to the strap.

It is therefore another embodiment, aspect, objective, and advantage of the present invention to provide a tourniquet device, wherein the pulley member includes a roller portion rotatably coupled to a hinged portion.

It is therefore another embodiment, aspect, objective, and advantage of the present invention to provide a tourniquet device, wherein the pulley member includes a clamping mechanism configured to allow the strap to pass substantially freely in one direction and substantially preventing the strap from passing in the opposite direction.

It is therefore another embodiment, aspect, objective, and advantage of the present invention to provide a tourniquet device, further comprising means for attaching for releasably securing the distal end portion of the strap to the tourniquet device.

It is therefore another embodiment, aspect, objective, and advantage of the present invention to provide a tourniquet device, wherein the means for attaching is configured to allow for incremental loosening or tightening of the tourniquet device and reattachment of the distal end to the strap at each incremental pressure decrease or increase as applied to such limb.

It is therefore another embodiment, aspect, objective, and advantage of the present invention to provide a tourniquet device, wherein the device is configured for wearing or storing in a form selected from a member of the group consisting of a belt, a backpack strap, integrated as part of a piece of clothing, and integrated as part of a vehicle.

It is therefore yet another embodiment, aspect, objective, and advantage of the present invention to provide a tourniquet device, wherein the device can function as a device selected from the group consisting of a suspensory device, restraint, and securing device for a splint.

It is therefore yet another embodiment, aspect, objective, and advantage of the present invention to provide a tourniquet device, further comprising:

hook-and-loop fabric on the strap, the hook-and-loop fabric having both hooks and loops at every part so that any part of the hook-and-loop fabric may attach to any other part.

It is therefore yet another embodiment, aspect, objective, and advantage of the present invention to provide a method of applying a tourniquet device to apply pressure to an artery in a limb, comprising the steps of:

encircling such limb above the point of blood loss with a tourniquet device comprising:

a strap having a proximal end portion and a distal end portion,

a buckle assembly connected to the proximal end portion of the strap; and

a pulley assembly slidably coupled to the strap and movable between the proximal end portion and the distal end portion, the pulley assembly having a pulley assembly support and a pulley member rotatably coupled to the pulley assembly support;

looping the distal end portion of the strap through the buckle assembly to reverse directions;

wherein the buckle assembly of step ii) further comprises a second pulley member rotatably coupled to a frame and wherein the looping of the distal end portion of the strap is through the second pulley member;

then looping the distal end portion of the strap through the first pulley member; whereby a pulley having a two-to-one force multiplication is formed by the first and second pulleys and the strap passing between them;

rotating the strap about such limb until the first pulley member is directly over such artery;

applying sufficient pressure to constrict blood flow through such artery by pulling the distal end of the strap away from the pulley assembly to tension the strap, transferring circumferential pressure to such limb and direct pressure to such artery; and

releasably securing the distal end portion to an means for attaching.

It is therefore yet another embodiment, aspect, objective, and advantage of the present invention to provide a tourniquet method, wherein the pressure applied to such limb and artery is incrementally reduced or increased by releasing the distal end portion from the means for attaching and resecuring the strap to the means for attaching at an incremental position equivalent to a reduction or increase in pressure to such limb.

It is therefore yet another embodiment, aspect, objective, and advantage of the present invention to provide a tourniquet method, wherein the means for attaching is hook-and-loop fabric on the strap, the hook-and-loop fabric having both hooks and loops at every part so that any part of the hook-and-loop fabric may attach to any other part.

While the tournaquet devices and methods will be described for the sake of grammatical fluidity with functional explanations, it is to be expressly understood that the claims, unless expressly formulated under 35 USC 112, or similar applicable law, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112, or similar applicable law. The invention can be better visualized by turning now to the following drawings wherein like elements are referenced by like numerals.

### BRIEF DESCRIPTION OF THE DRAWINGS

FIG. 1 is a perspective view of a tourniquet device secured around a limb in accordance with embodiments of the new technology.

FIG. 2 is a front view of the tourniquet device shown in FIG. 1 worn as a belt in accordance with the new technology.

FIGS. 3A and 3B is an enlarged isometric view of a pulley assembly configured in accordance with embodiments of the new technology.

FIG. 4 is an enlarged top view of a buckle assembly configured in accordance with embodiments of the new technology.

FIG. 5 is an enlarged back view of a portion of the tourniquet device shown in FIG. 1 in accordance with embodiments of the new technology.

FIG. 6A is an isometric view of a buckle assembly configured in accordance with other embodiments of the new technology, and FIG. 6B is a top view of the buckle assembly shown in FIG. 6A.

FIG. 7 is a side view of a tourniquet device configured in accordance with further embodiments of the new technology.

FIG. 8 is a side view of a simplified true pulley showing basic principles of mechanical engineering. This is PRIOR ART.

### DETAILED DESCRIPTION

Glossary

There are 6 different categories of simple machines: the inclined plane, the wheel/axle, the lever, the true pulley, the screw and the wedge, which were long ago categorized and understood. This may be verified at such sources as: Sciencing.com: http://sciencing.com/list-five-types-pulleys-8673129.html, or at LIVE SCIENCE, http://wvvw.livescience.com/49106-simple-machines.html as well. This is not a new classification either, the ancient history of the simple machines may be verified at https://social-la-wiki.wikispaces.com/Greek+Inventions or https://prezi.com/awargsdp5tuu/the-history-of-single-machines/. Hero of Alexandria's work “Book On Raising Heavy Weights” illustrates pulleys in antiquity.

The pulley may be seen to have several key features: a rope used to apply tension (unlike a lever, inclined plane, etc), two pulleys which change the direction of the rope by 180 degrees TWICE, and so on.

The present invention teaches, among other features, a pulley used to tighten a tourniquet which cuts off arterial blood flow. The word “pulley” is used throughout the present application. However, it has two different meanings, and regrettably, the two meanings must BOTH be used for clarity, even in the claims.

FIRST USAGE OF “PULLEY”: technically, a simple wheel, with the axle mounted and a rope passing around 180 degrees of the circumference of the wheel is NOT a pulley, but rather a “sheave” or “block”. Nonetheless, the word pulley is the easiest description of a single wheel with a rope about it. A single such “pulley” in fact confers NO mechanical advantage: when the user pulls the rope by a displacement Y against force of X/2, the load on the other side of the single wheel/block/sheave/pulley also moves by the exact same increment: displacement Y due to the same force of X/2. However, the single sheave is nonetheless commonly called a “pulley” and for clarity will be called as such herein. If this is an issue during prosecution, the word “block” may be substituted at a later point in prosecution, being more technically correct but less useful for communication.

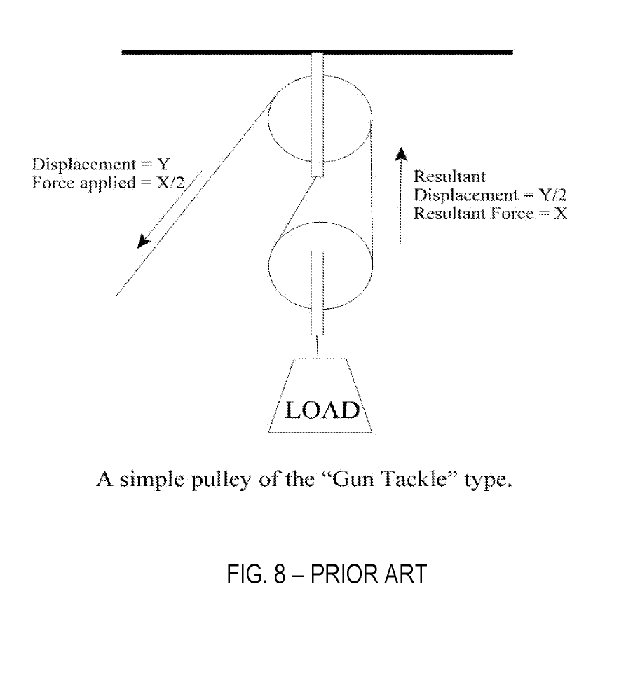

SECOND USAGE OF THE WORD PULLEY: The true pulley. FIG. 8 illustrates a “true pulley” which offers 2:1 mechanical advantage. A single true pulley requires the use of “two pulleys”, meaning two blocks. It may be seen that when the user of a true pulley pulls the rope by the displacement of Y, with a force of X/2, the resultant displacement is only Y/2, however, the user's mechanical advantage exerts a force X (double the original force) on the load to be moved.

Tourniquets require a surprising amount of force to be effective. Under battlefield conditions, first aid conditions or other stress conditions it may be hard to exert such force. In particular, the force necessary to close an artery, which is normally well inside of the human body, can be quite high. The femoral artery for example sits against the large muscles of the human thigh.

Hook-and-loop fabric as used herein may refer to fabric in which one area has hooks and another part has loops, however, the type in which every part of the hook-and-loop fabric has both hooks and loops is preferred (Omni-tape® made by the original Velcro® company is an example of this type), as this type of fabric can stick to itself at any part to any part: it is no longer necessary to match a hooks only part to a loops only part.

End Glossary

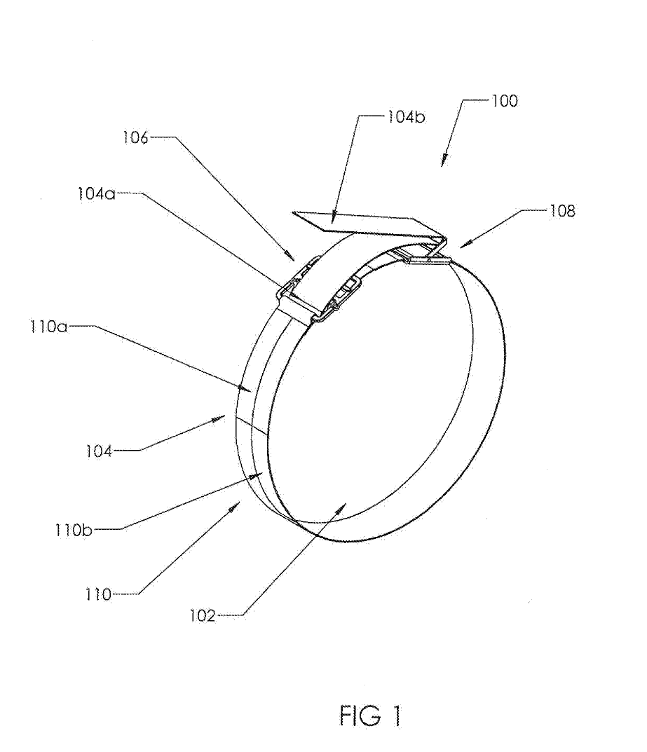

The present technology is directed to tourniquet devices and associated methods of use and manufacture. A tourniquet device configured in accordance with several embodiments of the disclosure can include, for example, a strap having a proximal end portion and a distal end portion, a buckle assembly connected to the proximal end portion, and a pulley assembly movably coupled to the strap. In operation, the strap can be routed around a limb and through the buckle assembly. From there, the strap can reverse direction such that the strap routes around at least a portion of the limb a second time and passes through the pulley assembly. A user can then apply a force to the distal end portion of the strap to tighten the tourniquet device around the limb, and the distal end portion of the strap can be secured in place with an attachment mechanism once a sufficient occlusive pressure is applied to the limb. When not in use, the tourniquet device can be worn (e.g., as a belt or restraint, integrated into clothing, etc.), such that the tourniquet device is conveniently stored, easily accessible in emergencies, and quickly applied to injuries. It will be appreciated that several of the details set forth below are provided to describe the following embodiments in a manner sufficient to enable a person skilled in the relevant art to make and use the disclosed embodiments. Several of the details described below, however, may not be necessary to practice certain embodiments of the new technology. Additionally, the new technology can include other embodiments that are within the scope of the claims but are not described in detail with reference to FIGS. 1-7.

Many of the details, dimensions, angles and other features shown in the Figures are merely illustrative of particular embodiments of the invention. For example, the shapes of various elements and angles are not drawn to scale, and some of these elements are arbitrarily enlarged and positioned to improve drawing legibility. Further, the particular shapes of the elements as drawn are not intended to convey any information regarding the actual shape of the particular elements, and have been solely selected for ease of recognition in the drawings. Accordingly, other embodiments can have other details, dimensions, angles and features without departing from the spirit or scope of the present disclosure. In addition, identical reference numbers are used in the Figures to identify identical or at least generally similar elements. To facilitate the discussion of any particular element, the most significant digit or digits of any reference number refers to the Figure in which that element is first introduced. For example, element 110 is first introduced and discussed with reference to FIG. 1.

FIG. 1 is a perspective view of an arterial tourniquet device 100 being tensioned around a person's limb 102 in accordance with embodiments of the new technology. The tourniquet device 100 can include a strap 104, a buckle assembly 106, and a pulley assembly 108. The buckle assembly 106 can be secured to a proximal end portion 104a of the strap 104, and the pulley assembly 108 can be movably coupled to the strap 104 such that the pulley assembly 108 can move along a length of the strap 104 to accommodate differently sized limbs (e.g., arms, legs, etc.). For example, the pulley assembly 108 can be moved closer to the buckle assembly 106 for smaller limbs (e.g., arms), and farther from the buckle assembly 106 for larger limbs (e.g., legs). Additionally, the pulley assembly 108 can be positioned along the strap 104 to gain additional leverage when tensioning the tourniquet device 100.

The strap 104 can have a diameter D that can apply sufficient pressure to constrict or occlude blood flow from the limb 102, while preventing the strap 104 from constricting the limb to the point of injury and/or amputation. In some embodiments, for example, the strap 104 can have a diameter of three inches. In other embodiments, the strap 104 can have a smaller diameter, a larger diameter, or vary in diameter. The strap 104 can be made from woven nylon (e.g., webbing), leather, plastic, rubber, cotton, and/or another suitable material that can withstand tension forces sufficient to cut off blood flow.

The tourniquet device 100 can further include an attachment mechanism 110 that can releasably secure a free portion (e.g., the distal end portion 104b) of the strap 104 to the tourniquet device 100. As shown in FIG. 1, the attachment mechanism 110 can be strips of Velcro® (one strip visible in FIG. 1) spaced along a circumference of the strap 104 at 110a and 110b. In other embodiments, the attachment mechanism can be a snap, a clip, and/or another suitable attachment mechanism that can releasably secure the strap 104 to the tourniquet device 100.

In some embodiments, the pressure applied by the tourniquet device 100 to the limb 102 can be slowly and/or partially released to prevent damage to the limb 102 (e.g., gangrene), sudden blood loss, and/or renewed bleeding. Thus, the attachment mechanism 110 can be configured to allow for incremental loosening of the tourniquet device 100 and reattachment at each incremental pressure decrease. For example, the attachment mechanism 110 can include a plurality of fasteners (e.g., Velcro® Omnitape® strips) spaced around the strap 104 at 110a and 110b, or comprising the entire strap or a substantial length thereof, and/or a releasable fastener (e.g., a clip) that allow the strap 104 to be re-secured to the attachment mechanism 110 during each incremental pressure decrease. Similarly, the attachment mechanism 110 can also be used to incrementally increase pressure.

In an emergency, the tourniquet device 100 can completely encircle the limb 102 above the point of blood loss (e.g., a bullet hole), and preferably with the entire device 100 rotated about the limb as much as is necessary to align one of the two buckle/pulley assemblies with a major blood vessel. The distal end portion 104b of the strap 104 can be routed through the buckle assembly 106, looped around the buckle assembly 106, where the strap 104 can reverse direction. The strap 104 can at least partially encircle the limb 102 for a second time such that the distal end portion 104b can be routed through and around the pulley assembly 108. The distal end portion 104b of the strap can be pulled away from the pulley assembly 108 to tension the strap 104. The tension can transfer to the limb 102 such that the tourniquet device 100 applies circumferential pressure to the limb 102 to constrict or occlude blood flow from above the tourniquet device 100. In addition, the pulley assembly 108 located over the blood vessel will have a portion which pushes into the flesh of the limb of the patient and directly pressure the blood vessel, aiding occlusion. (In the case of assembly 106, the pulley member itself may in alternative embodiments be designed and configures so that it too digs downward, into the opening holding the limb (102), however this is not the presently preferred embodiment.) Assembly 108 (in particular the tray/pulley support member 212, has a pulley member which pulls upward and thus torques/levers the edges of the assembly downward into the flesh.

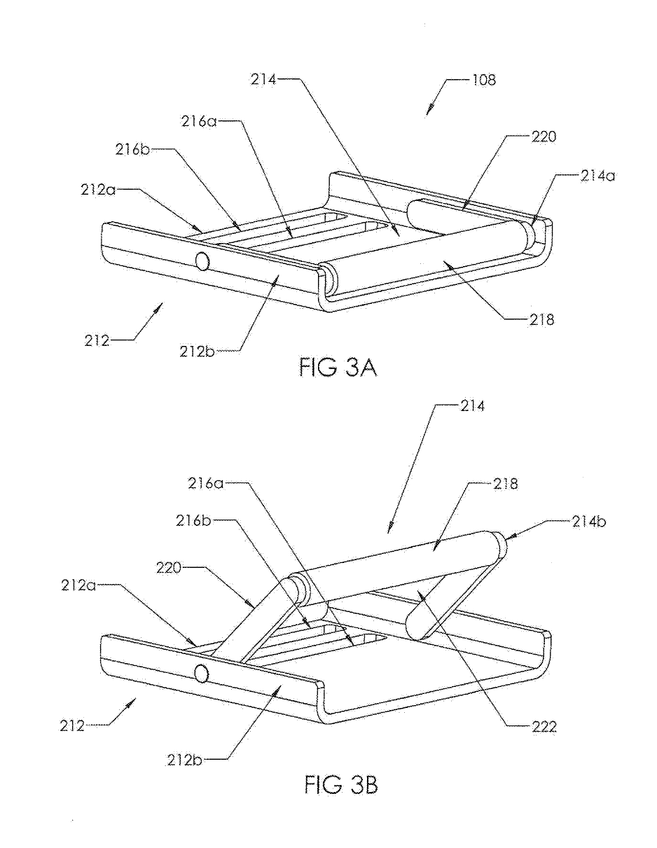

In other alternative embodiments the tray 212 may be attached to the pulley at one edge (rather than near the middle as shown in FIGS. 3A/3B) with a pinch bar/axle extending across the tray and no apertures (216a, 216b are thus omitted) to grip the strap when the pulley is under tension upward away from the tray. In this embodiment, the pulley member in fact levers the tray 212 edge into the artery in a triangle or “V” shape quite effectively.

The pulley assembly 108 can approximately double the force exerted by a user on the tourniquet device 100 to the limb 102. For example, if an occlusive pressure is X, the user need only exert approximately X/2 to adequately tension the tourniquet device 100 around the limb 102 and cut off blood flow. Once a desired pressure is reached (e.g., enough pressure to stop bleeding), the attachment mechanism 110 can releasably secure the distal end portion 104b of the strap 104 to the tourniquet device 100. If desired, the pressure to the limb 102 can be released incrementally by reducing the tension on the strap 104 and re-securing the strap 104 to the attachment mechanism 110. In operation, the tourniquet device 100 multiplies the force exerted by the user such that the tourniquet device 100 can be applied with one hand and can be used constrict or occlude the user's own blood flow. This can be especially advantageous when the user is injured and/or lacks the strength or dexterity necessary to achieve occlusive pressure. Advantageously, the tourniquet 100 device can also be used as a sling for a broken arm. For example, a user can encircle the strap 104 around his or her neck, and secure the strap at a desired length using the buckle assembly 106, the pulley assembly 108, and/or the attachment mechanism 110. The user can then support his or her arm with the strap 104. Additionally, the tourniquet device 100 can also be used to secure a splint in place by wrapping the strap 104 around the limb and the splint and tightening the tourniquet device 100 using the pulley assembly 108 and/or the buckle assembly 106. Moreover, the tourniquet device 100 can find a wide variety of other uses consistent with the present invention and of those skilled in the art, including: being tied around a person's body and/or limbs to restrain the person (e.g., in combat situations), used to create an emergency evacuation litter, as a gun strap to carry a gun and can be used to create a backpack or as backpack straps or compression straps on a backpack. The tourniquet device 100 can also be used as tie down straps, towing straps, as a climbing harness, as a fixed loop for helicopter rescue, as an animal (e.g. dog) harness, animal leash, animal collar or animal restraint.

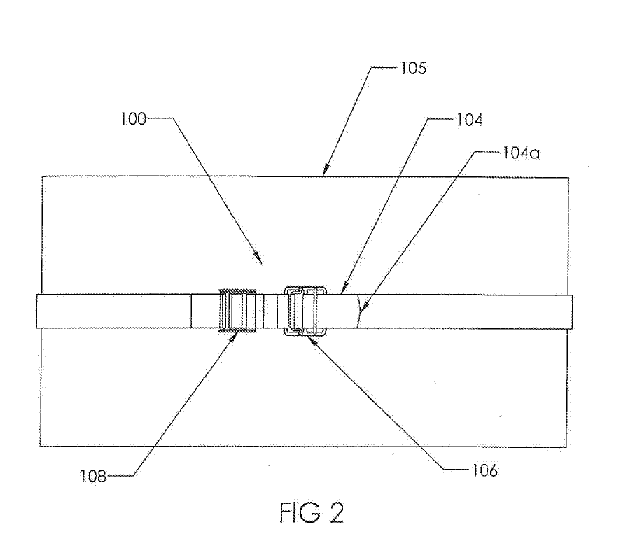

When the tourniquet device 100 is not in use, the tourniquet device 100 can be worn for convenient storage and quick accessibility. For example, FIG. 2 is a perspective view of the tourniquet device 100 shown in FIG. 1 worn as a belt in accordance with the new technology. The tourniquet device 100 can be looped around a torso 105, through belt loops 103, and can be secured using the buckle assembly 106. The pulley assembly 108 can be moved along the length of the strap 104 such that the pulley assembly 108 to secure a loose end (e.g., the distal end portion 104b) in place. In an emergency, the tourniquet device 100 can quickly be removed from the torso 105 and secured around the limb 102. Thus, when the tourniquet device 100 is not in use, the tourniquet device 100 can serve an additional functional purpose (e.g., a belt) that does not require additional storage. In other embodiments, the tourniquet device 100 can be worn and/or used with another article of clothing or accessory, such as a back pack strap.

In further embodiments, the tourniquet device 100 can be integrated into clothing at select locations (e.g., upper limb portions). For example, the tourniquet device 100 can encircle an upper thigh portion in each leg of a pair of pants and a detachable fabric portion can cover the strap 104, the buckle assembly 106, and/or the pulley assembly 108 for unobtrusive storage. In an emergency, a wearer must only remove the fabric portion to access and employ the tourniquet device 100 on his or her leg. Thus, the tourniquet device 100 can be conveniently stored around a limb for quick application in emergencies. In still further embodiments, the tourniquet device 100 can be integrated into or used as a vehicle restraint (e.g., a seat belt) and detached from the vehicle for use as a tourniquet.

FIG. 3 is an enlarged isometric view of the pulley assembly 108 shown in FIG. 1. The pulley assembly 108 can include a support member 212 and a pulley member 214 (shown at a first position 214a and at a second position 214b) rotatably connected to the support member 212. NOTE again that the pulley member 214 is not a complete mechanical pulley but is rather an individual pulley/sheave/block. The support member 212 can include a first surface 212a and a second surface 212a opposite the first surface 212a. In the embodiment illustrated in FIG. 3, the support member has a substantially rectangular shape that has generally similar dimensions as the strap 104 (shown in FIG. 1) and curved portions that can enclose a portion of the pulley assembly 108. In other embodiments, the support member 212 can have different shapes suitable for supporting the pulley member 214.

As shown in FIG. 3, the support member 212 can in some embodiments further include apertures 216 (identified individually as a first aperture 216a and a second aperture 216b) extending through the support member 212. The apertures 216 can be sized to receive the strap 104, and slidably attach the pulley assembly 108 to the strap 104. In some embodiments, for example, the distal end portion 104b of the strap 104 can be routed from the second surface 212a, through the first aperture 216a, across a portion of the first surface 212a between the apertures 216, and through the second aperture 216b. In other embodiments, the strap 104 can be woven through the apertures 216 in the opposite direction and/or the support member 212 can include more or less apertures 216 sized to receive the strap 104.

The slidable attachment provided by the apertures 216 or other mechanisms allows the pulley assembly 108 to move along the length of the strap 104 such that the position of the pulley assembly 108 can change to accommodate differently sized limbs and/or provide better leverage to the user. When the pulley assembly 108 is at a desired position, the apertures 216 can substantially restrict the movement of the pulley assembly 108 to prevent it from moving as the tourniquet device 100 is tensioned. Additionally, as described with reference to FIG. 2, the slidable attachment provided by the apertures 216 allows the pulley assembly 108 to be moved when the tourniquet device 100 is used as a belt to secure loose, free end portions of the strap 104. In other embodiments (discussed previously in reference to FIG. 1) the apertures 216 may be omitted and the pulley member hinged portion 220 may be hinged much closer to one edge.

In other embodiments, the support member 212 can include other fasteners that allow the pulley assembly 108 to move along the length of the strap 104 to accommodate differently sized limbs and/or torsos. For example, in some embodiments, the support member 212 can include a clip having a push button that, when engaged, allows the pulley assembly 108 to move along the length of the strap 104 and, when released, substantially secures the pulley assembly 108 in the desired position. In further embodiments, the support member 212 can include clips, snaps, and/or other suitable fasteners that slidably and/or releasably secure the pulley assembly 108 to the strap 104. In still further embodiments, the pulley assembly 108 can be detachable from the tourniquet device 100 when not in use and secured with a clip and/or other suitable fastener in an emergency.

The pulley member 214 can include a roller portion 218 rotatably coupled to a hinged portion 220 such that the roller portion 218 can spin 360° about the hinged portion 220. As shown in the embodiment in FIG. 2, the hinged portion 220 can extend through the roller portion 218 to serve as a spindle for the roller portion 218. In other embodiments, the hinged portion 220 can be rotatably coupled to end portions of the roller portion 218. The hinged portion 220 can be rotatably coupled to the support member 212 with hinges, pins, and/or other suitable devices that allow the pulley member 214 to rotate.

In some embodiments, the pulley member 214 can be biased by a spring force and/or include a locking mechanism to move the pulley member 214 to specified positions. For example, the pulley member can be locked at an angle (e.g., 45°, 90°) away from the support member 212 while tensioning the tourniquet device 100 for optimal leverage on the strap 104. As another example, the pulley member 214 can be biased toward the support member 212 such that the pulley member 214 is compactly stored when not in use. In other embodiments, the pulley member 108 can further include a cover (not shown) that encloses the pulley member 214 when it is not in use to prevent the pulley member 214 from catching on surroundings.

This bias to higher angles is especially useful to help provide targeted pressure directly to a specific blood vessel thereunder. By increasing the angle of the pulley members relative to the assemblies, a wider “V” or triangle shape is created and a greater arterial pressure may be applied.

As shown in FIG. 3B, the hinged portion 220 and the roller portion 218 can form an opening 222 such that the strap 104 can be routed around the roller portion 218. The roller portion 218 can have a length L that is at least the diameter D (shown in FIG. 1A) of the strap 104 such that the roller portion 218 can provide a contact point for the strap 104 when the tourniquet device 100 is tensioned. The roller portion 218 can be made from materials having low coefficients of friction to allow the strap 104 to smoothly glide over the roller portion 218. In some embodiments, for example, the roller portion 218 can be made from smooth plastics, metals, and/or other substantially frictionless materials. Additionally, the entire pulley member 214 (e.g., the roller portion 218 and the hinged portion 220) can be made from a material able to withstand the forces applied to the pulley assembly 108 without substantial deformation. For example, the pulley member 214 can be made from metals, strong plastics, and/or other suitable materials.

As illustrated by the arrow in FIG. 3A, the pulley member 214 can rotate with respect to the support member 212 from the first position 214a to the second position 214b. In the first position 214a, the pulley member 214 can be substantially flush and/or parallel to a first surface 212a of the support member 212. The pulley member 214 can be rotated to the first position 214a for compact storage when the tourniquet device 100 is not in use. Additionally, the pulley member 214 can be placed in the first position 214a to secure a free end portion of a strap 104 under the pulley member 214. In the second position 214b, the pulley member 214 can rotate away from the first surface 212a of the support member 212. The pulley member 214 can be moved to the second position 214b during use as a tourniquet such that the strap 104 can be routed through the opening 222 and around the roller portion 218 to tension the strap 104 around a limb (e.g., the limb 102 described in FIG. 1). In other embodiments, the pulley member 214 can rotate beyond the second position 214b, and/or rotate freely 180° from the first position 214b to be proximate to the apertures 216 at the first surface 212a. In further embodiments, the pulley member 214 can rotate more or less with respect to the support member 212.

In selected embodiments, the pulley assembly 108 can also include a clamp mechanism that can (1) tension the tourniquet device 100 tighter around a limb as a user pulls more of the strap 104 through the clamp mechanism, and (2) retain that tension when the user releases the strap 104. For example, the clamp can allow the strap 104 to pass substantially freely in one direction (e.g., tightening the tourniquet device 100) and can substantially prevent the strap 104 from passing in the opposite direction (e.g., loosening the tourniquet device 100). In additional embodiments, the clamp mechanism can also include a release feature, such as a button, knob, or other suitable manual or electronic release, that can be used to loosen the strap 104 from the clamp mechanism.

The pulley member 214 can reduce the amount of force required to attain a requisite pressure. For example, the pulley member 214 can reduce the force required by approximately half. This is especially helpful when the user applying the tourniquet device 100 is wounded himself and/or lacks the strength to apply the appropriate amount of pressure to slow or occlude bleeding.

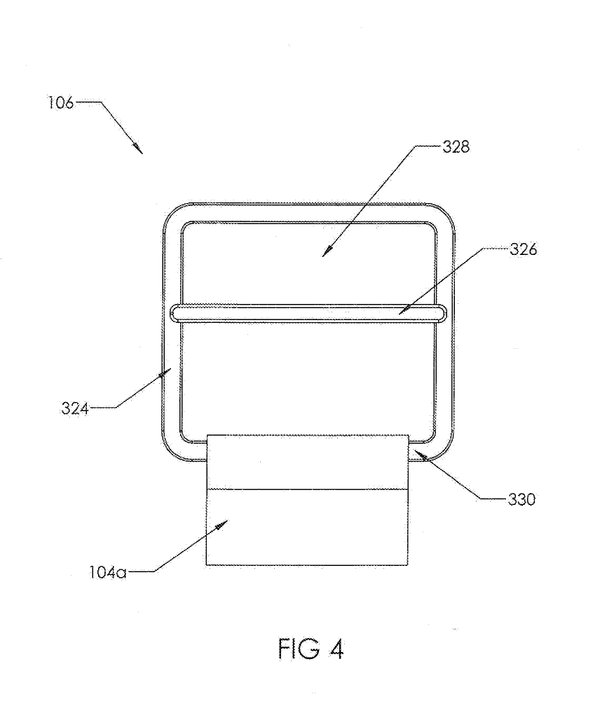

FIG. 4 is an enlarged top view of the buckle assembly 106 shown in FIG. 1. The buckle assembly 106 can include a frame 324 and a post 326. As shown in FIG. 4, the frame 324 can form an opening 328 such that the proximal end portion 104a of the strap 104 can be secured around a back portion 330 (shown in hidden lines) of the frame 324. The proximal end portion 104a can be attached around the back portion 330 with thread, snaps, glue, and/or other suitable fasteners that can secure the proximal end portion 104a to the frame 324. In other embodiments, the proximal end portion 104a of the strap 104 can be integrally formed.

The post 326 can extend across the opening 328, substantially parallel to the back portion 330 of the frame 324. In some embodiments, the post 326 can be slidably attached to the frame 324 such that the post 326 can adjust the width of the opening 328 to secure the strap 104. In other embodiments, the post 326 can be fixedly attached to the frame 324 and/or the frame 324 and the post 326 can be a single piece. The frame 324 and the post 326 can be made from semi-rigid and/or rigid materials that can withstand the force applied to the tourniquet device 100 to occlude blood flow. For example, in some embodiments, the frame 324 and the post 326 can be made from steel alloys, strong plastics, and other suitable materials.

In operation, the distal end portion 104b of the strap 104 can be routed through the opening 328, and the strap 104 can be looped around the post 326 and/or the frame 324 such that the strap 104 encircles a limb. If used as a belt, the distal end portion 104b can be woven around the buckle assembly 106 like a conventional box-frame belt buckle.

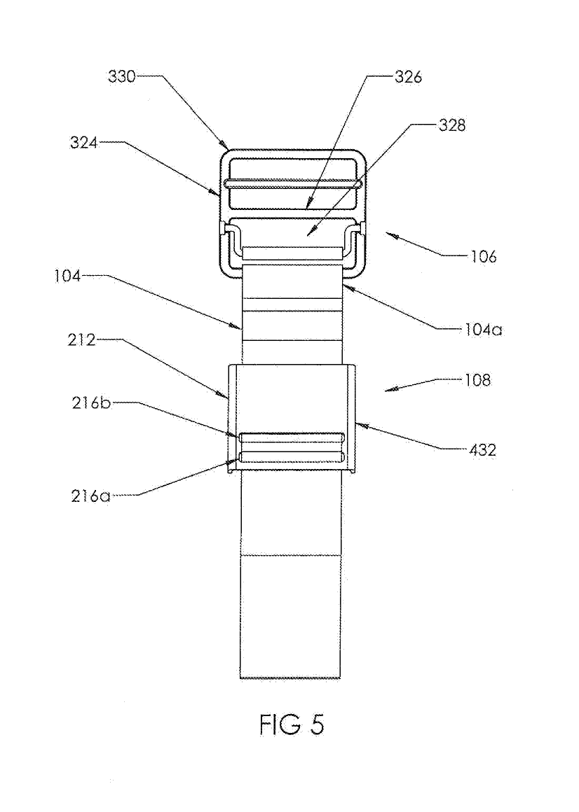

FIG. 5 is an enlarged back view of the buckle assembly 106 and the pulley assembly 108 shown in FIGS. 1-4. In the illustrated embodiment, the proximal end portion 104a of the strap 104 is secured to the back portion 330 of the frame 324. The post 326 extends across the opening 328 formed by the frame 324 such that the distal end portion 104b (not visible) of the strap 104 can be woven through the buckle assembly 106. The pulley assembly 108 can be slidably attached to the strap 104 by weaving the strap through the apertures 216. In the embodiment illustrated in FIG. 3, the hinged portion 220 of the pulley member 214 is attached to the support member 212 with pins 432 (not visible) that allow the pulley member 214 to pivot with respect to the support member 212. Additionally, FIG. 1 shows the attachment mechanism 110 includes strips of Velcro® (shown individually as a first attachment mechanism 110a and a second attachment mechanism 110b) spaced along the strap 104 such that the tourniquet device 100 can be incrementally tightened or loosened.

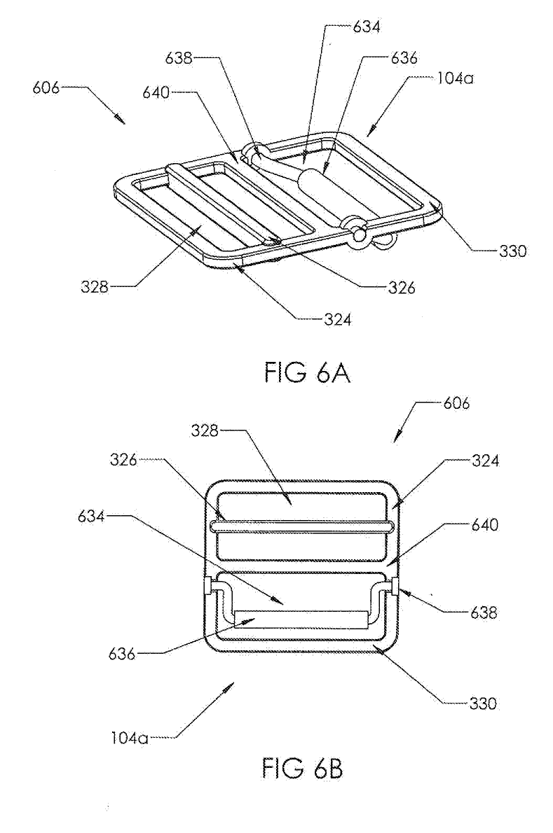

FIGS. 6A and 6B are isometric and top views, respectively, of a buckle assembly 606 configured in accordance with other embodiments of the new technology. The buckle assembly 606 includes features generally similar to the buckle assembly 106 described with reference to FIG. 4. For example, the buckle assembly 606 includes the frame 324, the post 326, and the opening 328 across which the post 326 extends. The back portion 330 of the frame 324 can be coupled to the proximal end portion 104a of the strap 104.

In the embodiment illustrated in FIGS. 6A and 6B, the buckle assembly 606 can further include a second pulley member 634. NOTE yet again that the pulley member 634 is not actually a true pulley (it is a block/sheave/etc) BUT, with the strap running between the two pulley members on the two assemblies, a true pulley is formed and true mechanical advantage is achieved in a tourniquet. The second pulley member 634 can include features generally similar to the pulley member 214 described in FIG. 2. For example, the second pulley member 634 can include a roller portion 636 and a hinged portion 638 that are at least generally similar to the roller and hinged portions 218 and 220 described above. In the embodiment shown in FIGS. 6A and 6B, the second pulley member 634 further includes a reinforcement member 640 that extends across the opening 328 and provides support for the second pulley member 634 when the strap 104 is tensioned around it. The reinforcement member 640 can be substantially parallel to the post 326. In other embodiments, the second pulley member 634 second does not include the reinforcement member 640, while in further embodiments the buckle assembly 606 includes additional reinforcement members 640.

As shown in FIG. 6A, the second pulley member 634 can extend downwardly away from the frame 324 such that the second pulley member 634 biases the buckle assembly 606 away from a limb (e.g., the limb 102 described above). The biased pulley member 634 can prevent the buckle assembly 606 from pinching skin on the limb during tensioning of the tourniquet device 100. When the buckle assembly 606 is not in use, the second pulley member 634 can retract toward the frame 324 as shown in FIG. 6B. In some embodiments, the second pulley member 634 can be biased further away from and/or closer toward the frame 324 than shown in FIGS. 5A and 5B. In other embodiments, the second pulley member 634 can rotate about the frame 324, and can be secured in desired positions using suitable locking mechanisms. In further embodiments, the second pulley member 634 can be freely hinged about the frame 324 and/or another portion of the buckle assembly 606. In still further embodiments, the second pulley member 634 can be attached in a fixed position to a portion of the buckle assembly 606.

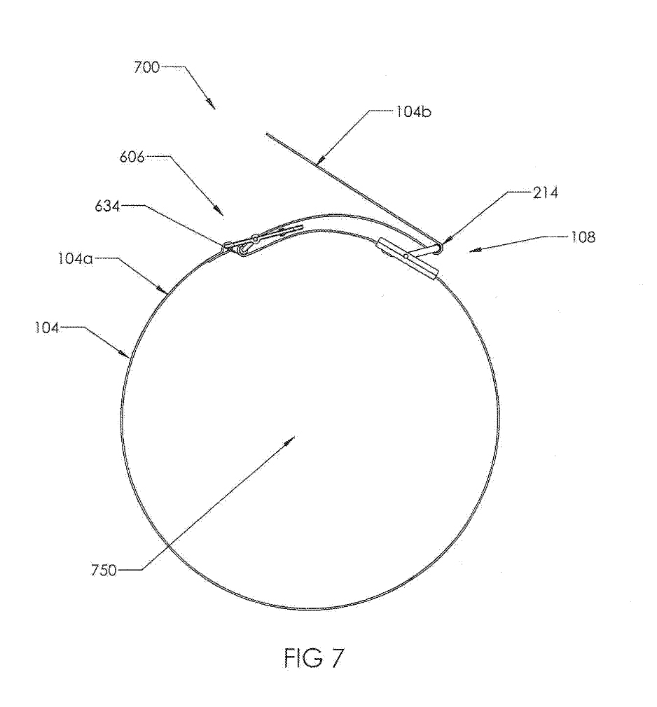

FIG. 7 is a side view of the tourniquet device 700 in accordance with further embodiments of the new technology. The tourniquet device 700 can include features generally similar to the tourniquet device 100 described in FIG. 1. For example, the tourniquet device includes the strap 104, the pulley assembly 108 having the support member 212 and the pulley member 214. Additionally, the tourniquet device 700 includes the buckle assembly 606 described in FIGS. 6A and 6B that includes a pulley. Thus, the tourniquet device 700 shown in FIG. 7 includes two pulleys: one on the pulley assembly 108 and another on the buckle assembly 606. Advantageously, this double-pulley tourniquet device 100 can require even less force from a user to constrict or occlude blood flow.

As shown in FIG. 7, the tourniquet device 700 can form an opening 750 through which a limb can extend. The distal end portion 104b of the strap 104 can be routed through the buckle assembly 606 and around the second pulley member 634. The second pulley member 634 can bias the buckle assembly 606 away from the skin to ensure the limb is not pinched when tensioning the tourniquet device 700. The addition of the second pulley member 634 to the tourniquet device 700 can also reduce the amount of force exerted by the user to adequately tension the tourniquet device 700. For example, the strap 104 can slide easily over the buckle assembly 606, creating less friction, and thus requiring less force to obtain occlusive pressure.

As further shown in FIG. 7, the distal end portion 104b can be redirected to surround at least a portion of the limb a second time. The distal end portion 104b can then extend through the opening 222 formed by the pulley member 214 of the pulley assembly 108. The tourniquet device 700 can be tensioned around the limb by pulling on the distal end portion 104b of the strap 104. In the embodiment illustrated in FIG. 7, the pulley member 214 is locked in place an angle away from the support member 212 when the tourniquet device 700 is tensioned to provide better leverage.

As discussed in the GLOSSARY, FIG. 8 is a block diagram of a simple pulley, illustrating the nature of a pulley.

From the foregoing, it will be appreciated that specific embodiments of the present technology have been described herein for purposes of illustration, but that various modifications may be made without deviating from the technology. For example, in the embodiments illustrated in FIG. 1, the pulley assembly 108 is positioned near the buckle assembly 106. However, the pulley assembly 108 can be positioned anywhere along the strap 104 to achieve maximal leverage. Additionally, padding can be added to the tourniquet devices 100, 700 (e.g., on the strap 104, on the support member 212) described in FIGS. 1-7 to provide additional comfort for an injured person. Certain aspects of the new technology described in the context of particular embodiments may also be combined or eliminated in other embodiments. For example, the tourniquet devices 100, 700 described in FIGS. 1-7 can include additional pulley members and/or additional pulley assemblies. Further, while advantages associated with certain embodiments of the new technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A tourniquet device, for a primary use applying pressure to an artery in a patient's limb and a secondary use worn as a belt looped around a wearer's torso, the tourniquet device comprising:
a strap having a proximal end portion and a distal end portion;
a first buckle assembly connected to the proximal end portion of the strap;
a second pulley assembly slidably coupled to the strap and movable between the proximal end portion and the distal end portion, the second pulley assembly having first and second apertures passing therethrough, the second pulley assembly located at a distance from the first buckle assembly;
the second pulley assembly having a pulley assembly support and a first pulley member rotatably coupled to the pulley assembly support and biased away from the pulley assembly support;
the first buckle assembly having a frame for connection to the proximal end portion of the strap and forming an opening wherein at least one post extends across the opening;
the first buckle assembly further having a second pulley member rotatably coupled to the frame, the second pulley member rotating away from the frame;
the strap passing through the first and second apertures of the second pulley assembly and then about the second pulley of the first buckle assembly;
whereby a pulley having a two-to-one force multiplication is formed by the first and second pulleys and the strap passing between them;
and further whereby an opening is formed through which in a first such usage, such artery in such limb may extend and be encircled and in a second such usage such tourniquet device may be worn as a belt.

2. The tourniquet device of claim 1, wherein the first pulley extends away from the pulley assembly support and the opening; whereby when tightened it exerts torque which forces the pulley assembly support into a part of the limb directly under the first pulley, and when the part of the limb directly under the first pulley is such artery, such artery is occluded.

3. The tourniquet device of claim 2, wherein the second pulley member includes a pulley reinforcement support extending across the opening and provides support for the second pulley member.

4. The tourniquet device of claim 1, wherein the pulley assembly support is configured to include at least one aperture for slidably attaching the pulley assembly to the strap.

5. The tourniquet device of claim 1, wherein the pulley member includes a roller portion rotatably coupled to a hinged portion.

6. The tourniquet device of claim 1, wherein the pulley member support includes a clamping mechanism configured to allow the strap to pass substantially freely in one direction and substantially preventing the strap from passing in the opposite direction.

7. The tourniquet device of claim 1, further comprising means for attaching for releasably securing the distal end portion of the strap to the tourniquet device.

8. The tourniquet device of claim 7, wherein the means for attaching is configured to allow for incremental loosening or tightening of the tourniquet device and reattachment of the distal end to the strap at each incremental pressure decrease or increase as applied to such limb.

9. The tourniquet device of claim 1, wherein the device is configured for wearing or storing in a form selected from a member of the group consisting of a belt, a backpack strap, integrated as part of a piece of clothing, and integrated as part of a vehicle.

10. The tourniquet device of claim 1, wherein the device can function as a device selected from the group consisting of a suspensory device, restraint, and securing device for a splint.

11. The tourniquet device of claim 1, further comprising:
hook-and-loop fabric on the strap, the hook-and-loop fabric having both hooks and loops at every part so that any part of the hook-and-loop fabric may attach to any other part.

12. A method of applying a tourniquet device to apply pressure to an artery in a limb, comprising the steps of:
i) encircling such limb above the point of blood loss with a tourniquet device comprising:
a strap having a proximal end portion and a distal end portion,
a buckle assembly connected to the proximal end portion of the strap; and
a pulley assembly slidably coupled to the strap and movable between the proximal end portion and the distal end portion, the pulley assembly having a pulley assembly support and a pulley member rotatably coupled to the pulley assembly support;
ii) looping the distal end portion of the strap through the buckle assembly to reverse directions;
wherein the buckle assembly of step ii) further comprises a second pulley member rotatably coupled to a frame and wherein the looping of the distal end portion of the strap is through the second pulley member;
iii) then looping the distal end portion of the strap through the first pulley member;
whereby a pulley having a two-to-one force multiplication is formed by the first and second pulleys and the strap passing between them;
iv) rotating the strap about such limb until the first pulley member is directly over such artery;
v) applying sufficient pressure to constrict blood flow through such artery by pulling the distal end of the strap away from the pulley assembly to tension the strap, transferring circumferential pressure to such limb and direct pressure to such artery; and
vi) releasably securing the distal end portion to an means for attaching.

13. The method of claim 12, wherein the pressure applied to such limb and artery is incrementally reduced or increased by releasing the distal end portion from the means for attaching and resecuring the strap to the means for attaching at an incremental position equivalent to a reduction or increase in pressure to such limb.

14. The method of claim 13, wherein the means for attaching is hook-and-loop fabric on the strap, the hook-and-loop fabric having both hooks and loops at every part so that any part of the hook-and-loop fabric may attach to any other part.
